(19) United States
(12) Patent Application Publication
(10) Pub. No.: US 2008/0226648 A1
(43) Pub. Date: Sep. 18, 2008

(54) Methods for Treating Stress and Affecting Biological Immune Systems

(76) Inventors: Francis Chi, Kowloon (HK); Jinxian Xu, Shanghai (CN); Gary Kwan Po Wong, Kowloon (HK)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/585,790

(22) PCT Filed: Jan. 13, 2005
(86) PCT No.: PCT/IB2005/000745

§ 371 Date: Jul. 26, 2006

Related U.S. Application Data
(60) Provisional application No. 60/536,016, filed on Jan. 13, 2004.
(60) Provisional application No. 60/599,331, filed on Aug. 5, 2004.

(51) Int. Cl. A61K 39/395; A61K 31/145; A61P 37/00; A61P 43/00

(52) U.S. Cl. 424/141.1; 514/665

(57) ABSTRACT

The subject invention provides materials and methods for treating stress and/or augmenting immune response. More specifically, the present invention provides methods for the treatment and/or prevention of stress-related physiological responses; the alleviation of stress-related symptoms; as well as the prevention or delay in development of stress-related complications. The present invention further provides biologically-active compounds that can cause the thymus and spleen to increase in size and. cause an increase in villi length and goblet cell production in mucosae. Specifically exemplified herein is the use of a cysteamine compound to modulate immune responsiveness and/or treat stress.

### CROSS-REFERENCE TO A RELATED APPLICATION

This application claims the benefit of U.S. Provisional Application Ser. Nos. 60/536,016 and 60/599,331, filed Jan. 13, 2004 and Aug. 5, 2004, respectively.

### BACKGROUND OF THE INVENTION

The immune system is a highly complex, biological system that requires the cooperation of a large number of different cell types. The systems of the body that make up the immune system network are variously categorized as belonging to the hematopoietic system, the reticuloendothelial or phagocytic system, and the lymphatic system.

The lymphatic system is made up of lymphocytes, and is responsible for the overall regulation of the immune system and for the production of antibodies. Lymphocytes can be concentrated within organs or can form a more or less diffuse lymphoid tissue, both of which collectively constitute the lymphatic system. Primary lymphatic organs (also known as “central tissues”), such as the thymus and bone marrow, are the major sites of lymphopoiesis. Secondary lymphoid organs and tissues (also known as “peripheral tissues”), such as the spleen, lymph nodes, lymphoid formations associated with the mucosae (or “MALT” for mucosal associated lymphoid tissue), Peyer's patches, and palatine tonsils, are those sites within which lymphocytes can interact with one another or with a foreign substance or organism (also known as an “antigen”). In addition, a large number of lymphocytes can be found in the mucosa of the stomach, of the small intestine, of the colon, of the bronchi and of various other organs.

Although there are many classes of lymphocytes, T-lymphocytes (or T-cells) and B-lymphocytes (or B-cells) make up a majority of the lymphocyte population. B-lymphocytes are generally responsible for the production of antibodies (immunoglobulin) in response to a challenge by a particular antigen. T-lymphocytes are responsible for the general regulation of the immune system and are also the principal mediators in cell-mediated immune responses.

All lymphocytes are ultimately derived from stem cells in bone marrow. These lymphocyte precursors are dispersed into the blood where they course through many organs. However, critical events take place in the thymus that imprint the lymphocytes with special functions and that regulate lymphocyte development into either T- or B-lymphocytes.

B-lymphocytes have significantly different biological functions from those of T-lymphocytes. While B-lymphocytes are involved in the final pathway of the humoral immune system (antibody production), T-lymphocytes encompass several subtypes of cells that play roles varying from immune system regulation to execution of the cytotoxic immune system. In addition to being responsible for antibody production, B-cells have some ability to engulf and “present” the antigen to a specific T-cell (also known as a T-helper cell) to stimulate the immune system.

T-lymphocytes are formed in the thymus from lymphoblasts that have left the bone marrow. The thymic cortex is rich in lymphocytes of all sizes. These thymocytes are not morphologically distinguishable from lymphocytes in other tissues, but they are immature and antigenically identifiable by the presence of several cell surface antigens including the T antigen, which is a distinctive surface marker antigen that separates the T-lymphocyte from the B-lymphocyte.

The spleen, which is located under the left side of the diaphragm, receives blood from an artery off of the aorta. Nests of B-lymphocytes surround the blood vessels of the spleen. After passing through this intricate meshwork of tiny blood vessels, the blood continues to the liver.

The spleen serves at least four major physiologic functions. First, as part of the peripheral immune system, it clears the blood of microorganisms and particulate antigens and/or generates antigens to foreign substances. Second, it sequesters and removes excess, old and/or abnormal blood cells. Third, its vasculature is involved in the regulation of portal blood flow. Finally, it engages in hematopoiesis during development or when the bone marrow alone cannot produce sufficient blood cells.

The spleen consists of red pulp, which contains blood-filled sinuses and pulp cords lined by reticuloendothelial cells, and of white pulp, which is arranged around a central arteriole. The surrounding periarteriolar lymphoid sheath (PALS) contains both T- and B-lymphocyte areas. The T-lymphocyte area lies adjacent to the arteriole and consists of small, densely packed lymphocytes. Outside of the T-cell area is the follicular zone, which contains B-lymphocytes, and germinal centers, which are made up of B-cells and macrophages. The white pulp is surrounded by a marginal zone containing specialized, antigen-presenting macrophages and B-cells.

The intestine is the organ richest in lymphocyte cells, which circulate in blood vessels and lymph vessels, or which collect and multiply in the small intestine (major lymphocytic tissues in the small intestine known as Peyer's patches). These lymphoctyic cells are a component of MALT, which is subdivided into gut-associated lymphoid tissues (“GALT”) situated in the intestine and into bronchial-associated lymphoid tissues (“BALT”) situated in the bronchi, and which has analogies with skin-associated lymphoid tissues (“SALT”) situated in the skin. GALT and BALT are interdependent and act in synergism to ensure the immune defense of the mucosa and to contribute towards the defense of the organism as a whole.

Unlike the lymph node, the Peyer's patches (PP) does not have a capsule of afferent lymphatics. The epithelium over the PP lacks the crypts and villi of normal gut epithelium and is referred to as follicle-associated epithelium (FAE) containing cells called M cells. These are the major route of antigen transfer into the PP, and allow for direct sampling of antigen from the gut lumen by pinocytosis. Antigen is transported from the epithelium and presented to immunocompetent B-cells, macrophages and dendritic cells in the underlying area. The colon has similar lymphoid arrangements called the lymphoid follicles. Lymphoid follicles are not identical to PP, but also have specialized epithelium containing M cells, and probably function as antigen presenting sites.

Underneath the epithelium there is a tissue called the lamina propria, which forms the core of the villus and is densely infiltrated with lymphocytes bearing homing receptors, which selectively bind to the mucosal lymphoid high endothelium. B-cells comprise about 50% of the lymphocytes in the lamina propria of the gut, whereas the other half of lymphocytes are T-cells. In the normal intestine, most of the B-cells in the lamina propria are IgA+, although IgM-, IgG- and IgD-expressing cells are also found. Most of the immunoglobulin secreted into the intestine is IgA, and half of that is IgA-2, in contrast to the lymph nodes where most of the secreted IgA is of the IgA-1 isotype. The abundance of IgA antibodies is considered crucial for immunological homeostasis within the lamina propria. IgA antibodies lack potent effector functions such as complement activation, and may therefore block non-specific biological amplification mechanisms triggered by locally produced or serum-derived IgG antibodies.

Anatomically, the thymus is situated in the anterior thoracic cage over the heart. A bilobed organ, the thymus is composed of epithelial cells and other structural cells that divide it into a complex assembly of continuous lobes, each of which is heavily laden with lymphocytes. The thymus is nourished and drained by the vascular and lymphatic systems, and innervated by the autonomic nerves.

Embryologically, the thymus emerges from the third and fourth branchial pouches. The human thymus is a fully developed organ at birth and weights 15 to 20 grams. By puberty it weighs 40 grams, after which it atrophies or involutes, becoming less significant structurally and functionally. Atrophy of the thymus with age is a characteristic of all species, which is associated with aging, and the cessation of growth. The incidence of age related diseases increases as the thymus shrinks and thymus-dependent immunity decreases. This age-associated decrease in thymic weight, called “involution,” is accompanied by changes in the thymic structure and a general decline in thymic function.

Thus, the thymus is normally active only during the early years of life. During those years, the thymus supplies T-lymphbcytes that will serve the animal for the rest of its life. In certain diseases, such as rheumatoid arthritis, the thymus may regain some activity during adult life. This demonstrates that the adult thymus retains the capacity to function and that involution is not necessarily permanent. At least partial function might be restored if appropriate agents are available.

Transient involution of the thymus may also occur as a consequence of a stress or infection. Thymic involution may be controlled hormonally; castration slows involution while injection of corticosteroid hormones accelerates involution. Numerous studies have demonstrated that the thymic involution associated with increasing age parallels a reduction of T-lymphocyte-mediated immunity and increased incidence of diseases associated with aging. Many diseases and treatments can accelerate involution of the thymus; virtually none are known to enhance growth of the thymus or reverse involution.

Studies have demonstrated that the thymus in the normal adult is slowly involuting, which affects thymus function. Moreover, in the normal adult, killed T-lymphocytes cannot be replaced, thus leaving the patient vulnerable to subsequent infections. Especially striking are recent studies of the thymuses of deceased AIDS patients ranging in age from 10 months to 42 years. AIDS victims have profound thymic involution; much more extensive than in age-matched patients who died of other causes.

Another area where there is a need to re-establish the lymphatic system, and also the hematopoietic system, is in total body irradiation for treatment of leukemia. When a patient undergoes high dose total body irradiation, the entire immune system is compromised. The usual treatment after the irradiation is to perform a bone marrow transplant with marrow from a close relative. If the transplant is successful, the new marrow will produce new cells, thereby restoring both red blood cells and white blood cells to the body. However, this is a dangerous treatment that is successful in only a fraction of the cases. Localized radiation of tumors and several types of chemotherapy also produce suppression of T-cell mediated immunity.

In addition, there is a need for effective treatments for stress, which may be also be useful in augmenting and/or preventing any decrease in lymphatic organ function. Stress is defined as a physical, chemical, or emotional factor that causes bodily or mental tension and may be a factor in disease causation (see Merriam-Webster Medical Dictionary, Merriam-Webster, Inc. (2002)). Mental tension (or emotional stress) can occur when situations are considered difficult or unmanageable. Psychological stresses induced by restraint, confinement, sudden exposure to danger, shock and the like translate into physical stresses affecting one or more organs of the body. Bodily tension (or physical stress) refers to a physiological reaction of the body to any of the factors cited above. Physical stress such as exposure to heat or cold, injury including surgical injury, over-exertion and the like, result in abnormal functioning of body organs. Stress is now recognized as a major detrimental factor in many diseases such as cardiovascular disease, cancer, and immunological dysfunction (see Kodama, M. et al., “Does surgical stress cause tumor metastasis?” Anticancer Res, 12(5):1603-16 (1992); Habra, M. et al., “Type D personality is related to cardiovascular and neuroendocrine reactivity to acute stress,” J Psychosom Res., 55(3):235-45 (2003); and Uchino, B. et al., “Individual differences in cardiac sympathetic control predict endocrine and immune responses to acute psychological stress,” J Pers Soc Psychol., 69(4):736-43 (1995)).

Physiologic responses to all stresses are the same; only the intensity of the response and whether or not any given response will be evoked are highly individual. Acute stress, such as that resulting from a trauma, robbery, or loud noise produces a physiologic response that quickly disappears, after which the body returns to its normal, unstressed state. Chronic stress, caused for example by a divorce, an unpleasant boss, or lack of money, is more insidious; the physiologic response endures and the body fails to return to its normal, baseline state. Studies have shown that being in a continuous state of stress can lead to tissue changes (i.e., adrenal hypertrophy, gastrointestinal ulceration, thymic and lymphoid atrophy) and may even culminate in death (Selye H A, The Stress of Life, New York, N.Y.: McGraw-Hill (1976)).

A variety of physiological functions occur as a result of stress, including changes in immune function, hormone levels, enzymes, and gastrointestinal function. In particular, “stress hormones” such as norepinephrine, epinephrine, and cortisol are released into the circulation to prepare the body for a “flight or fight” response after a stressful event. The effects of stress on a mammal normally manifest themselves in an increase in body temperature, along with a charge in hemodynamic parameters, including an increase in heart rate and an increase in blood pressure. For patients already suffering from elevated blood pressure (i.e., hypertension), the effects of stress can be particularly dangerous since hypertension is a major risk factor for cardiovascular disease.

Stress and emotions associated with stress are important risk factors in the development of cardiovascular problems (Kawachi, I. et al., “Symptoms of anxiety and risk of coronary heart disease,” The Normative Aging Study. Circulation, 90:2225-2229 (1994)). For example, when comparing men reporting the lowest levels of worry (an emotion associated with stress) against men reporting the highest level of worry, men with highest level of worry had a multivariate adjusted relative risk of 2.41 (95% confidence interval: 1.40-4.13) for nonfatal myocardial infarction and of 1.48 (95% confidence interval: 0.99-2.20) for total coronary heart disease (nonfatal myocardial infarction and fatal coronary heart disease). See Kubzansky, LD et al., “Is worrying bad for your heart? A prospective study of worry and coronary heart disease in the Normative Aging Study,” Circulation, 95:818-824 (1997).

Moreover, research indicates that a bout of acute stress in virtually any form will cause, at the very least, a temporary decrease in functioning of the immune system. Whereas in the case of chronic stress, a continued decline in immune system function is often observed (Irwin, M. et al., “Reduction of immune function in life stress and depression,” Biol Psychiatry, 27:22-30 (1990)). Specifically, stress has a detrimental effect on the ability to maintain optimal levels of natural killer (NK) cell cytotoxic activity (Irwin, M. et al., “Plasma cortisol and natural killer cell activity during bereavement,” Biol Psychiatry, 24:173-178 (1988); Sieber, W. J. et al., “Modulation of human natural killer cell activity by exposure to uncontrollable stress,” Brain Behav Immun, 6:141-156 (1992)), which plays a vital role in immune system surveillance against viral-infected and cancer cells.

Stress also has a significant influence on the balance of intestinal microflora (see Huis Veld J H, “Gastointestinal flora and health in man and animal,” Tijdschr Diergeneeskd, 116:232-239 (1991) [article in Dutch]). Research has demonstrated that, “the composition of the [intestinal micro]flora was not significantly affected by drastic changes in diet, but statistically significant shifts in the proportions of some species [of microflora] were noted in individuals under conditions of anger or fear stress.” See Moore, W E et al., “Some current concepts in intestinal bacteriology,” Am J Clin Nutr, 31:S33-S42 (1978). An imbalance in intestinal microflora population can eventually present gastro-intestinal disorders/conditions (i.e., irritable bowel syndrome or ulcers).

Drugs have been produced that are efficient in the control and treatment of stress and impairment. Unfortunately, few, if any, of those drugs produce both an etiopathogenic action as well as symptomatological action.

Therefore, what is needed is a safe and effective method of stimulating immune responsiveness (i.e., by restoring or enhancing T- and B-lymphocyte function) in the lympathic system, in particular to stimulate growth in the thymus and spleen and to enhance villi length and goblet cell production in mucosae. What is also needed is a means for reducing stress. In doing so, lymphatic organ activity may be maintained and/or augmented.

However, to date, there is no effective treatment that will cause the thymus to reverse the process of involution and produce new T-lymphocytes. Nor has a successful method been provided to enhance splenic function and/or enhance mucosa protection against pathogens as well as treat stress. Further, there has been no disclosure to date regarding the use of a cysteamine compound for ensuring optimal lymphatic organ mass as well as treating stress.

### BRIEF SUMMARY OF THE INVENTION

The subject invention provides materials and methods for treating stress and affecting biological immune systems in many different ways. Accordingly, the subject invention teaches the administration of at least a cysteamine compound to a patient.

In certain embodiments, the materials and methods of the subject invention enable increased bioactivity in lymphatic organs. For example, by utilizing the methods of the invention, a patient can enhance the ability of mucosa to combat and/or prevent the occurrence of immunological diseases/disorders. In addition, methods of the invention can increase mass (or retard the deterioration in mass) of certain lymphatic organs (i.e., thymus or spleen). Further, the invention is useful in the treatment of stress-related physiological responses; the alleviation of stress-related symptoms; the prevention, delay, or alleviation of stress-related complications (i.e., dampened immune function); as well as the enhancement of immunological responsiveness.

In one embodiment, a cysteamine compound is administered to a patient either alone or concurrently with agent(s) known to treat stress and/or stress-related symptoms and/or complications.

In accordance with the subject invention, administration of a cysteamine compound to a patient prior to experiencing a stressful event can help protect the patient from experiencing physiological responses induced by stress, or at least ensure that physiological responses to stress occur to a lesser extent than would be observed in the absence of the cysteamine compound. The administration of a cysteamine compound may even be beneficial to the patient's subsequent abilities to withstand the adverse effects of subsequently encountered stresses. For example, patients administered at least a cysteamine compound can be preconditioned for improved resistance and reaction to subsequently encountered stress.

In another embodiment, a cysteamine compound is administered to prevent and/or delay the development of stress-related symptoms and complications in patients. For example, stress-related symptoms or complications such as diminished mental and physical performance (i.e., chronic anxiety, nervous habits, insomnia, migraines, low back pain, nailbiting, skin picking, lack of sex-drive, loss of appetite, irrational behavior), hypertension, irritable bowel syndrome (IBS), obesity (caused from stress eating), drug and alcohol abuse, hair loss, Chronic Fatigue Syndrome, heartburn, gastric reflux, asthma, allergies, dampened immune function (i.e., chronic viral infections), breathing disorders, bruxism, premature aging, depression, hyperlididemia, cardiovascular disease, and diabetes, can be reduced through consumption, according to the subject invention, of a cysteamine compound.

In yet another embodiment, a cysteamine compound is administered to a patient diagnosed with an immunological disorder and/or condition to treat the disorder/condition as well as prevent and/or decrease the severity of complications related to the disorder/condition. In a related embodiment, a cysteamine compound is administered in combination with other known agents that are used to treat immunological disorders/conditions (i.e., autoimmune, inflammatory, proliferative and hyperproliferative diseases, cutaneous manifestations of immunologically mediated diseases).

Specifically exemplified herein is the use of a cysteamine compound to increase or maintain the mass of the thymus and/or spleen. Further, in a related embodiment, the use of a cysteamine compound increases villi length and goblet cell production in mucosa(e) in a patient to enhance immunological responsiveness. For example, common immunological disorders and/or conditions such as autoimmune disorders (i.e., Hashimoto's thyroiditis, pernidcious anemia, Addison's disease, rheumatoid arthritis, systemic lupus erythematosus, dermatomyositis, Sjogren's syndrome, dermatomyositis, lupus erthematosus, multiple sclerosis, myasthenia gravis, Reiter's syndrome, Graves disease), immune deficiency (i.e., AIDS), and multiple chemical sensitivity, can be treated through administration, according to the subject invention, of a cysteamine compound.

A therapeutically effective amount of a cysteamine compound for administration to a patient can be from about 0.1 mg to 3,000 mg/kg of body weight (BW) or an equivalent molar quantity of a cysteamine compound. Preferably, a daily dose of about 1 mg/kg BW to 30 mg/kg BW of a cysteamine compound, or an equivalent molar quantity of a cysteamine compound, is administered to an adult patient. In a more preferred embodiment, about 4 mg/kg BW to 18 mg/kg BW of cysteamine hydrochloride, or an equivalent molar quantity thereof, is administered daily to an adult patient.

### BRIEF DESCRIPTION OF DRAWINGS

FIG. 1 shows a metabolic pathway of cysteamine.

FIG. 2 shows cysteamine as a constituent of co-enzyme A.

FIG. 3 is a graphical illustration of cysteamine effect on IL-2 serum concentrations when administered in accordance with the subject invention.

Detailed Disclosure of the Invention

The subject invention provides materials and methods for treating stress and/or augmenting immune activity. The subject invention teaches the administration of a cysteamine compound to a patient suffering from stress and/or depressed immunity to treat and/or prevent stress-related physiological responses; alleviate stress-related symptoms; as well as prevent or delay the development of stress-related complications (ie., dampened immune function).

Certain embodiments of the invention include administering a cysteamine compound to patients diagnosed with immunological diseases/disorders to treat or prevent the occurrence of such diseases/disorders in a patient. In order to treat and/or prevent the development of immunological diseases/disorders, methods are provided by the subject invention for maintaining and/or preventing the deterioration in mass of thymus and/or spleen in a patient. In other embodiments, the present invention provides methods for affecting mucosae bioactivity, namely increasing villi growth and goblet cell activity. Further embodiments of the invention include establishing that a patient is under stress and administering a cysteamine compound to treat stress and stress-related symptoms and complications.

The term “treatment” or any variation thereof (i.e., treat, treating, etc.), as used herein, refers to any treatment of a patient diagnosed with a biological condition, such as stress or depressed immune activity, using the materials and/or methods of the invention. The term treatment, as used herein, includes: (i) preventing or delaying the presentation of symptoms associated with the biological condition of interest in an at-risk patient who has yet to display symptoms associated with the biological condition (i.e., preventing the presentation of depression in a patient who is suffering from stress); (ii) ameliorating the symptoms associated with the biological condition of interest in a patient diagnosed with the biological condition (i.e., eliminating tension headaches in a patient suffering from stress); (iii) preventing, delaying, or ameliorating the presentation of symptoms associated with complications, conditions, or diseases associated with the biological condition of interest (i.e., preventing, delaying or ameliorating the presentation of gastrointestinal disorders) in either an at-risk patient or a patient diagnosed with the biological condition; (iv) relieving the biological condition (i.e. causing regression of an immunological disorder); and/or (v) augmenting patient immunological activity (i.e., enhancing immunological responsiveness).

As used herein, “immunological diseases/disorders” includes both primary immunodeficiency (where the cause of the immune defect was/is unknown) and secondary immunodeficiency (where there is a recognized cause for the immune defect such as cancer, infection, and the like). An immunological disease/disorder of the invention includes conditions associated with previous treatment with chemotherapeutic agents, radiation, immunosuppressive and anti-inflanunatory drugs and dialysis; conditions such as severe combined immunodeficiency, congenital thymic aplasia, aplastic anemia, viral infections, chronic granulomatous disease and immune dysfunction associated with diabetes; adverse reactions to bone marrow or organ transplantation such as graft-versus-host disease; physical findings such as rashes, fevers, and adverse reactions indicative of, leukemias, lymphomas, inflammatory bowel disease or psoriasis. In addition, the term “immunological diseases/disorders” includes diseases classified as autoimmune in nature (see, Theofilopoulos, A., In: D. P. Stites, et al., eds., Basic and Clinical Immunology, Lange Medical Publications, Los Altos, Calif., 1988, which is incorporated in its entirety by reference).

The term “stress,” as used herein, refer to the physical, chemical, and/or emotional factor that causes bodily or mental tension, which may be a factor in disease causation. A “stressful event” or “stressful situation,” as used herein, refers to a psychological and/or physical occurrence that causes a patient to be affected by stress. Examples of stressful events include, but are not limited to, experiencing and/or anticipating the following: pressure to perform at work, school, or in sports, threats of physical violence, money worries, arguments, family conflicts, divorce, bereavement, unemployment, moving from a house, and alcohol and/or drug abuse.

The term “symptom(s)” as used herein, refers to common signs or indications that a patient is suffering from a specific condition or disease. For example, stress-related symptoms contemplated herein include, but are not limited to, uncontrollable shaking; hyperventilation; vomiting; triggering of an asthma attack; periods of irritability or anger; apathy or depression; constant anxiety; irrational behavior; loss of appetite; comfort eating; lack of concentration; lack of sex-drive; increased smoking, drinking, or recreational drug-taking; excessive tiredness; skin problems; aches and pains resulting from tense muscles (including neckache, backache, and tension headaches); increased pain from arthritis and other conditions; heart palpitations; irregular menstruation cycles (for women); constipation or diarrhea; dizziness; fainting spells; nail biting; frequent crying; sensation of pins and needles; increased tendency to perspire; and difficulty sleeping.

According to the present invention, symptoms of immunological diseases/disorders include, but are not limited to, oral ulcers; recurrent bacterial, viral, or fungal infections; dermatological rashes; arthritis; chronic diarrhea; nodular lymphoid hyperplasia; recurrent deep tissue swelling following minor trauma; telangiectases; asthma; rhinitis; eczema; and urticaria.

As used herein, the term “complication(s)” refers to a pathological process or event occurring during a disease or condition that is not an essential part of the disease or condition; where it may result from the disease/condition or from independent causes. For example, stress-related complications refer to medical/clinical problems that occur more often in patients who suffer from excessive, prolonged stress. As contemplated herein, complications associated with stress include, without limitation, increased susceptibility to bacterial infections (ie., tuberculosis, group-A streptococcal diseases), gastrointestinal disorders (i.e., stomach and duodenal ulcers, irritable bowel syndrome), cardiovascular diseases/disorders (i.e., hypertension, atherosclerosis, myocardial infarction, arrhythmia), asthma, rheumatoid arthritis, thyroid over-activity, and dermatological conditions (i.e., eczema, hives, psoriasis, or acne).

The term “patient,” as used herein, describes an organism, including mammals, to which treatment with the compositions according to the present invention is provided. Mammalian species that benefit from the disclosed methods of treatment include, but are not limited to, apes, chimpanzees, orangutans, humans, monkeys; and domesticated animals (i.e., pets) such as dogs, cats, mice, rats, guinea pigs, and hamsters.

“Concurrent administration” and “concurrently administering,” as used herein, includes administering a compound or therapeutic method (hereinafter referred to as the “therapeutic agent”) suitable for use with the methods of the invention. For example, for physical symptoms and/or complications that often accompany stress (i.e., gastro-intestinal conditions), a cysteamine compound can be concurrently administered with methods and/or pharmaceuticals known to be useful in treating such symptom/complication (i.e., antibiotics, acid blockers, antacids, proton pump inhibitors, cytoprotective agents, change in lifestyle such as cessation in smoking, etc.). In another example, a cysteamine compound can be concurrently administered with therapeutic agents known to be useful in treating immune deficiency disorders (i.e., AIDS).

By way of example, a compound for concurrent administration with a cysteamine compound of the invention can be provided in admixture with the cysteamine compound, such as in a pharmaceutical composition. Alternatively, the compound and cysteamine can be concurrently administered as separate compounds, such as, for example, separate pharmaceutical compositions administered consecutively, simultaneously, or at different times. Preferably, if the cysteamine compound and the therapeutic agent for treating/preventing stress and stress-related symptoms/complications are administered separately, they are not administered so distant in time from each other that the cysteamine compound and the therapeutic agent cannot interact.

Therapeutic agents that can be concurrently administered with a cysteamine compound in accordance with the present invention include, but are not limited to, corticosteroids such as prednisone and hydrocortisone; cytotoxic drugs such as azathioprine, cyclophosphamide, mycophenolate mofetil, and methotrexate; non-cytotoxic drugs such as ciclosporin and tacrolimus; nonsteroidal anti-inflammatory drugs such as ibuprofen and naproxen; COX-2 inhibitors such as celecoxib and rofecoxib; antimalarials such as hydroxychloroquine; plasma exchange; high-dose ivIg therapy; intravenous gamma globulin; monoclonal antibody (moAb) therapy; and therapies for use in treating stress such as counseling, psychotherapy, exercise, meditation, and massage.

As used herein, reference to a “cysteamine compound” includes cysteamine, the various cysteamine salts, which include pharmaceutically acceptable salts of a cysteamine compound, as well as prodrugs of cysteamine that can, for example, be readily metabolized in the body to produce cysteamine. Also included within the scope of the subject invention are analogs, derivatives, conjugates, and metabolites of cysteamine, which have the ability as described herein to treat stress and/or augment immune function. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

As contemplated herein, a cysteamine compound includes pantothenic acid. Pantothenic acid is a naturally occurring vitamin that is converted in mammals to coenzyme A, a substance vital to many physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and, concomitantly, cysteamine.

The term “pharmaceutically acceptable salt,” as used herein, refers to any salt of a cysteamine compound that is pharmaceutically acceptable and does not greatly reduce or inhibit the activity of the cysteamine compound. Suitable examples include acid addition salts, with an organic or inorganic acid such as acetate, tartrate, trifluoroacetate, lactate, maleate, fumarate, citrate, methane, sulfonate, sulfate, phosphate, nitrate, or chloride.

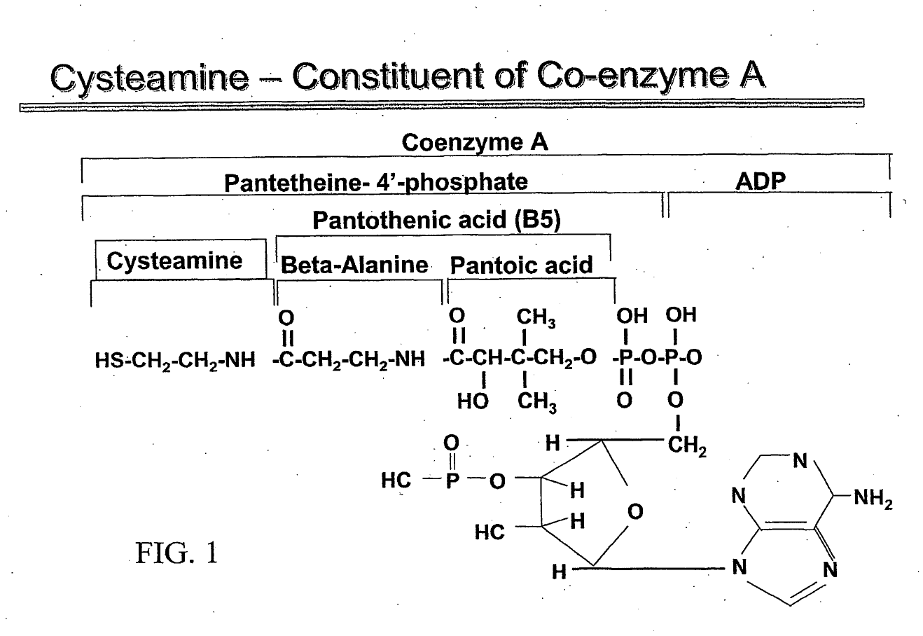

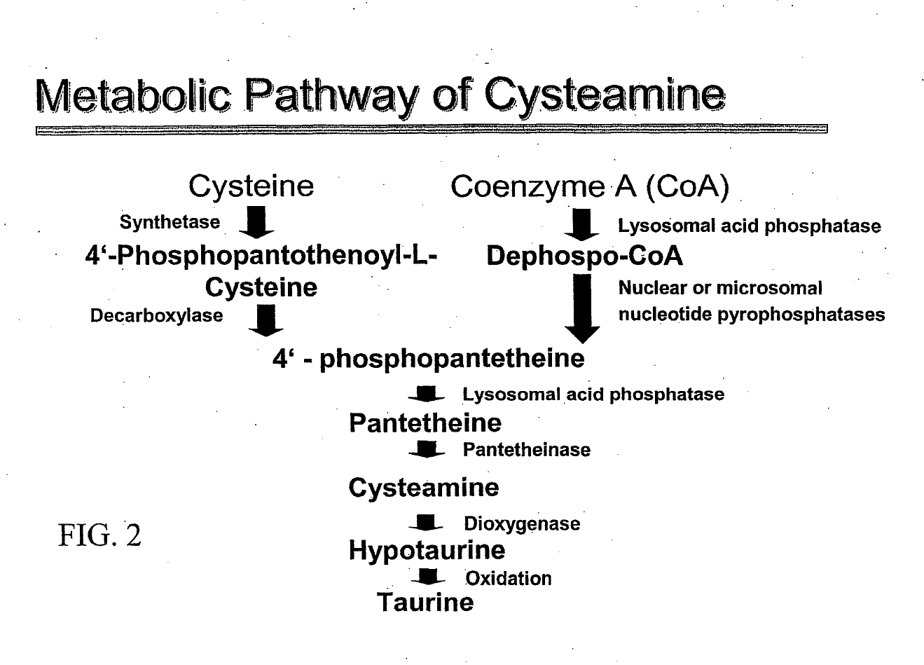

Accordingly, in one embodiment of the subject invention, the advantages of cysteamine, as set forth herein, can be achieved by promoting the endogenous production of cysteamine through natural metabolic process such as through the action of co-enzyme A or as a metabolite of cysteine (see FIGS. 1 and 2). This can be achieved by, for example, the administration of pantothenic acid.

The term “effective amount,” as used herein, refers to the amount necessary to elicit the desired biological response. In one embodiment of the invention, the effective amount of a cysteamine compound is the amount necessary to treat/prevent stress and/or stress-related symptoms/complications. In another embodiment, an effective amount of a cysteamine compound is the amount necessary to prevent the deterioration of thymus mass. In a further embodiment, an effective amount of a cysteamine compound is that necessary to ameliorate the severity of symptoms and/or complications associated with stress or decreased/dysfunctional lymphatic activity. The amelioration in symptom and/or complication severity may be a 5%, 10%, 15%, 20%, 25% 30%, 35%, 40%, 45%, 50%, 55%, 60%, 65%, 70%, 75%, 80%, 85%, 90%, 95%, 98% or 99% decrease in severity.

The compositions of the invention can be used in a variety of routes of administration, including, for example, orally-administrable forms such as tablets, capsules or the like, or via parenteral, intravenous, intramuscular, transdermal, buccal, subcutaneous, suppository, or other route. Such compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

The cysteamine compounds of the subject invention can be formulated according to known methods for preparing pharmaceutically useful compositions. Formulations are described in a number of sources, which are well known and readily available to those skilled in the art. For example, Remington's Pharmaceutical Science (Martin EW [1995] Easton Pa., Mack Publishing Company, 19^{th} ed.) describes formulations that can be used in connection with the subject invention. Formulations suitable for parenteral administration include, for example, aqueous sterile injection solutions, which may contain anti,oxidants, buffers, bacteriostats, and solutes, which render the formulation isotonic with the blood of the intended recipient; and aqueous and nonaqueous sterile suspensions, which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Administration of a cysteamine compound, in accordance with the subject invention, can be accomplished by any suitable method and technique presently or prospectively known to those skilled in the art. In a preferred embodiment, a cysteamine compound is formulated in a patentable and easily consumed oral formulation such as a pill, lozenge, tablet, gum, beverage, etc. In one embodiment, consumption of a cysteamine compound is taken at, prior to, or after, experiencing a stressful event. In another embodiment, a cysteamine compound is administered to a patient prior to, during, or after a decline in thymus and/or spleen mass.

Compositions of the invention comprise, as an active ingredient, an effective amount of the cysteamine and one or more non-toxic, pharmaceutically acceptable carrier or diluent. Examples of such carriers for use in the invention include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents.

To provide for the administration of such dosages for the desired therapeutic treatment, compositions of the invention will typically comprise between about 0.1% and 99%, of the total composition including carrier or diluent. The dosage used can be varied based upon the age, weight, health, or the gender of the individual to be treated.

Specifically exemplified herein is the use of a cysteamine compound to restore, maintain, and/or improve the performance of a patient's lymphatic system. The use of a cysteamine compound can also independently or concurrently treat stress and stress-related symptoms and complications. With respect to the lymphatic system, a cysteamine compound augments immune response by: increasing or maintaining organ mass, which enhances lymphocyte activity (such as T- and B-cell or antibody bioactivity); increasing villi growth along mucosal membranes; and/or increasing goblet cell activity to enhance non-specific immune defenses against antigens.

With respect to stress, a cysteamine compound lowers cortisol levels in a patient. Under stress, the body releases neurotransmitters (i.e., epinephrine, norepinephrine, serotonin) and cortisol to get the body back to a non-stressed state. In a chronic stress state, the neurotransmitters can become depleted and as a result, the lack of serotonin is directly related to poor mood and depression. Cortisol, however, does not get depleted and its continued presence in the body depresses mood. Long term exposure to cortisol leads to impaired memory, depressed immune function, central obesity, and development of chronic disease. Thus, administration of at least a cysteamine compound can treat stress-related physiological responses as well as treat and/or prevent stress-related symptoms and/or complications (i.e., decrease risk of chronic disease like heart disease and obesity due to long-term exposure to cortisol).

In certain embodiments, methods of the present invention are used for retarding the deterioration of and/or maintaining mass in the thymus and/or spleen of a patient to treat and/or prevent the development of immunological diseases/disorders including immune mediated cancers and hyperactive immune responses. Such immune mediated cancers may include lymphoreticular neoplasia, lymphoblastic leukemia, brain tumors, gastric tumors, plasmacytomas, multiple myeloma, leukemia, connective tissue tumors, solid tumors and lymphomas. Such hyperactive immune responses may include asthma/allergies and autoimmune diseases. Such allergies may include hay fever, atopic dermatitis, urticaria, perennial rhinitis, allergic conjunctivitis, pulmonary diseases, food allergies, skin allergies, anaphylaxis (e.g., associated upon exposure to blood products) and pollinosis.

In other embodiments, methods of the present invention are used for preventing the deterioration of and/or maintaining the mass in the thymus and/or spleen of a patient to treat and/or prevent the development of autoimmune diseases including, without limitation, type 1 diabetes, conventional organ specific autoimmunity, neurological disease, rheumatic diseases/connective tissue disease, autoimmune cytopenias, and related autoimmune diseases. Conventional organ specific autoimmunity may include thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo. Neurological diseases may include schizophrenia, Alzheimer's disease, depression, hypopituitarism, diabetes insipidus, sicca syndrome and multiple sclerosis. Such rheumatic diseases/connective tissue diseases may include rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjorgren's syndrome. Other autoimmune related diseases may include autoimmune uvoretinitis, glomerulonephritis, post, myocardial infarction cardiotomy syndrome, pulmonary hemosiderosis, amyloidosis, sarcoidosis, aphthous stomatitis, and other immune related diseases, as presented herein and known in the related arts. See, e.g., Berkow et al., eds, The Merck Manual, 16th edition, Merck and Co., Rahway, N.J., 1992, ages 303-364, 710-718, 1083, 1269, 1305-1377, 1338 1677-1684, and 2435-2438 which is incorporated herein in its entirety by reference.

Specifically exemplified herein is the use of cysteamine hydrochloride (and/or analogs, derivatives and prodrugs thereof) to treat and/or prevent the occurrence of an immunological disease/disorder in a patient.

According to the subject invention, the dosage of cysteamine administered to a patient to elicit a desired response is about 0.1 mg to about 3,000 mg/kg of body weight (BW) per day, or an equivalent molar quantity of a cysteamine compound thereof. In one embodiment, the desired response is treatment of stress, which can include (1) prevention of bodily and/or mental tension as a response to stress; (2) a reduction in the severity, duration, or intensity of bodily and/or mental tension as well as symptoms associated with stress; and (3) prevention, delay, or reduction in the severity, duration, or intensity of complications related to stress.

In another embodiment, the desired response is an effect on the patient's lymphatic system to improve a patient's immunological response. Contemplated effects on the lymphatic system include, but are not limited to, (1)increasing or maintaining lymphatic organ mass; (2) enhancing lymphocyte activity such as T- and B-cell or antibody bioactivity; (3) increasing villi growth along mucosal membranes; and (4) increasing goblet cell activity.

Preferably, a daily dose of about 1 mg/kg BW to 30 mg/kg BW of a cysteamine compound, or an equivalent molar quantity of a cysteamine compound, is administered to an adult patient to elicit a desired response. In a more preferred embodiment, about 4 mg/kg BW to 18 mg/kg BW of cysteamine hydrochloride, or an equivalent molar quantity thereof, is administered daily to an adult patient to elicit a desired response.

Following is an example that illustrates a procedure for practicing the invention. This example should not be construed as limiting. All percentages are by weight and all solvent mixture proportions are by volume unless otherwise noted.

### EXAMPLE 1

### Administration of Cysteamine Compound in Treating Stress

An experiment was conducted on sixteen castrated adult goats, which were randomly divided into control group (n=8) and treatment group (TG, n=8); housed separately; and fed with the same nutrient level. The TG was treated orally with a cysteamine compound (the cysteamine compound is preferably cysteamine hydrochloride, where a dose of 45 mg/kg of body weight was administered, with 30% of the 45 mg/kg dosage comprising the cysteamine compound) provided by Shanghai Walcom Bio-Chem Ltd). Both groups were subjected to rumen operation and duodenum operation.

This experiment was conducted in four phases: (a) Roughage phase pre-operation: from day 18 to day 14 before the operation, both groups of goats were fed with roughage (dried peanut stalk) and water; (b) Concentrate phase pre-operation: day 13 to day 1 before the operation (in total 13 days), both groups were fed with roughage and concentrate (50 g/head), and the TG was also treated with a cysteamine compound (using dosages described above) at the same time; (c) Rumen operation phase: after completion of the concentrate phase pre-operation, the rumen operation was performed to affix a fistula on the rumen, which was followed by 18 days of recovery; (d) Duodenum operation phase: 18 days after the rumen operation, a second operation was performed to affix a fistula on the duodenum, following which the goats were allowed to have 17 days of recovery to complete this experiment. The feeding diet during the operation was same as that of concentrate phase per-operation.

While conducting the experiment, blood samples were periodically collected. In general, blood was collected via the neck vein before morning feeding. Part of the blood from each sample was stored in tubes containing heparin for anti-coagulation. The remaining part of each blood sample was centrifuged and then stored the serum at −20° C. Blood samples were collected at the following times: (a) Day 4 of the Roughage phase pre-operation; (b) Day 13 of the Concentrate phase (one day before the operation, B1); (c) 10 days after the 1^{st} operation (rumen operation); and (d) 6 days and 17 days after the 2^{nd} operation (duodenum operation).

Serum IL-2, cortisol concentration and lymphatic transformation testing were also performed during the experiment. Radio-immuno assay (RIA) was used to test the serum IL-2 and cortisol concentration with respect to the suggested protocol. To perform lymphatic cells transformation test, the blood samples from both groups were treated as follows: 3 tubes/sample, with blood sample 100 ul/tube for PHA− (PHA−: without PHA) and 3 tubes/sample for PHA+(PHA+: each tube added with 50 ul PHA diluted solution); cells in both PHA− and PHA+ were cultured for total 72 hours. After 48 hours of cell culture, 1 uL ³H-TdR was added to each tube with and mixed with cells and incubated further for 24 hours. At the completion of the culturing process, the cells were lysed. The lysate from both PHA− and PHA+ tubes were transferred to scintillation bottles, dried at 80° C., then added with 5 ml scintillation solution. The solutions were stored overnight and tested for cpm. SI index implies the rate of lymphatic cells transformation induced by lectin, SI=cpm(PHA+)/cpm(PHA−).

IL-2 and cortisol testing kits were used. To prepare RPMI-1640 cell culture medium, RPMI-1640 powder was added with distillated water and filtered, and 20% Fetal Bovine Serum, Penicillin (100 U/ml) as well as Streptomycin (100 U/ml) was added. 2 ml culture medium solution was then added to each tube. In addition, diluted PHA, [Methy-³H] Thymidine with radioactivity 1 mci/m, and Scintillation solution: PPO (2,5, Diphenyloxazole,) 5 g POPOP(1,4bis [5-phenyloxazolyl] benzene) 0.3 g Dimethyl benzene: 800 mL; 100% ethanol: 200 ml were added to each tube.

All data from the experiment (see Table 1) were expressed in mean±SE and analyzed by Excel software for t-test analysis, where P<0.05 implies significant difference, and 0.05<P<0.15 implies changes. The symbol “§” in Table 1 denotes TG treated with Cysteamine. The symbol “*” in Table 1 denotes differences between groups with P<0.05; and the symbol “#” denotes differences between groups with 0.05<P<0.15. The symbol “^{ab}” in Table 1 denotes differences between periods within the same group with P<0.05; whereas the symbol “^{AB}” in Table 1 denotes differences between periods within the same group with 0.05<P<0.15.

TABLE 1
Cysteamine effect on IL-2 serum concentration in pre-, post-operation periods

| | Pre-operation | | After 1^{st} Operation | | |
|---|---|---|---|---|---|
| Group | Roughage | Concentrate | (ug/l) | After 2^{nd} Operation | |
| (n = 8) | phase | Phase | Day 10 ^{§} | Day 6 ^{§} | Day 17 ^{§} |
| Control | 1.21 ± 0.10 | 0.80 ± 0.05 ^{a A} | 0.65 ± 0.05 ^{B} | 0.49 ± 0.07 ^{b} | 0.52 ± 0.11 |
| Treatment | 0.92 ± 0.11 | 0.97 ± 0.15 | 1.32 ± 0.10 * ^{A} | 0.95 ± 0.06 * ^{B} | 1.02 ± 0.08 |

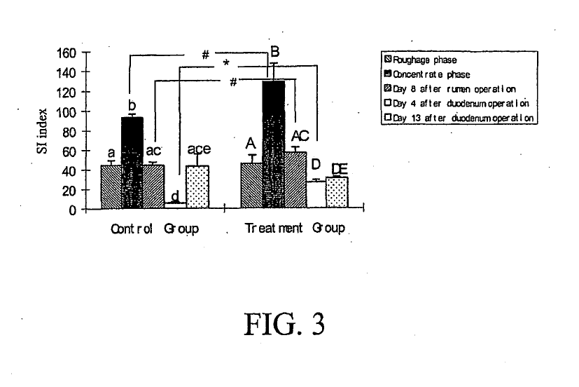

IL-2 concentration levels for each group taken at various times during the experiment are illustrated in FIG. 3. As compared to pre-operation IL-2 concentration, post-surgery IL-2 concentrations are lower in the control group. In particular, the lowering effect was observed in the control group after the 1^{st} surgery (p=0.14), and with a significant lowering effect after the 2^{nd} surgery (0.8±0.05 vs 0.49±0.07ug 1⁻¹), P<0.05). In contrast, the IL-2 concentration in TG showed no significant difference between pre- and post-operation. The symbols illustrated in FIG. 3 represent the following: differences between groups, where * is P<0.05; # is 0.05<P<0.15; and the difference between periods within the same group is symbolized with ^{ab/AB}, where P<0.05.

Before treatment with a cysteamine compound (in Roughage phase), serum IL-2 concentration levels between the control group and TG were not significantly different. In the Concentrate phase before the first operation, the IL-2 concentration levels in TG (0.80±0.05 vs 0.97±0.15 ug 1⁻¹) after treatment with a cysteamine compound were increased, but not significantly when compared with that of the control group (P=0.11). After the first operation, TG IL-2 concentration levels were higher than the control group by 90-100% (0.65+0.05 vs 1.32+0.10 ug 1⁻¹, 0.49±0.07 vs 0.95±0.06ug 1⁻¹), which is a significant difference (P<0.05). After 17 days from the 2^{nd} operation, the IL-2 concentration levels no longer fluctuated and the difference in IL-2 concentration levels between the two groups had disappeared.

In the Roughage phase pre-operation, there was no difference between the two groups in lymphatic cells transformation rate (40.32±5.23 vs 43.47±4.33). But in the Concentrate phase, the TG treated with a cysteamine compound increased in lymphatic cells transformation rate (90.2±7.41 vs 132.4±20.3), in which the difference was not significant when compared with the control group (P=0.14).

Both surgical operations caused the control group and the TG lymphatic cells transformation rate to decrease (P<0.05), where the degree of decrease in the TG was significantly smaller than that of the control group (P<0.05). Six days after the 2^{nd}/duodenum operation, the lymphatic transformation rate of the control group decreased down to 12% of the rate before the operation, while the rate of TG lymphatic cell transformation had increased by 3.5 fold (P<0.05) when compared to the rate of the control group. In the following days, the lymphatic transformation rate started to recover (control group P<0.05) and the difference between the two groups disappeared (P>0.05).

The cortisol concentrations of for each group taken at various times during the experiment are provided in Table 2 below. Cortisol concentrations of the two groups before the operation were substantially similar (20.51±4.1 for the control group versus 20.32±4.0 ug 1⁻¹ for the TG). Changes in cortisol concentration levels in the TG before and after the 1^{st} operation were slight. In contrast, cortisol concentration levels in the control group after 6 days from the 2^{nd} operation increased (P=0.08). In some instances, the cortisol concentration levels in the control group reached levels higher than the TG (P=0.11). The symbol “§” in Table 2 designates TG treated with Cysteamine. The symbol “*” in Table 2 designates a difference in cortisol concentration levels between groups with P<0.05. The symbol “^{ab}” designates a difference in cortisol concentration levels between periods within the same group with P<0.05. The symbol “#” designates a difference in cortisol concentration levels between periods within the same group with 0.05<P<0.15.

TABLE 2
Cysteamine effect on cortisol serum concentration in pre-, post-operation periods

| | Pre-operation (ng/ml) | | After 1^{st} operation | After 2^{nd} operation | |
|---|---|---|---|---|---|
| Group | Roughage | Concentrate | (ug/l) | (ug/l) | |
| (n = 8) | phase | phase ^{§} | Day 10 ^{§} | Day 6 ^{§} | Day 17 ^{§} |
| Control | 26.61 ± 3.2 | 20.5 ± 4.1 ^{a} | 27.21 ± 5.1 ^{a} | 30.21 ± 3.5 | 22.01 ± 4.2 |
| Treatment | 21.2 ± 2.2 | 20.3 ± 4.0 | 20.9 ± 3.7 | 21.2 ± 5.2 # | 20.7 ± 5.8 |

### EXAMPLE 2

### Administration of Cysteamine Compound and Immune Response in Murine Model

Male C57BL/6N mice were used in Example 2, 28 mice in total. The 28 mice were used for pretest for 7 days before the treatment began. Two groups of 14 mice were then created. As illustrated in Table 3, 12 mice from group 1 were orally administered a cysteamine compound, 27% (w/w) cysteamine hydrochloride, at a dosage of 40 mg/kg or 80 mg/kg of body weight. Each group contained six replicates and each replicate contained 8 mice. The treatment period was for 30 days.

TABLE 3
Experimental Model

| | |
|---|---|
| Control | Saline |
| Treatment 1 | Cysteamine hydrochloride-40 mg/kg BW |
| Treatment 2 | Cysteamine hydrochloride-80 mg/kg BW |

Lymphocyte transformation rate in plasma, white blood cell (WBC) count, natural killer cell activity, Plasma IL-2 (Kit provided by Jinginei Biotech) and IL-6 (Kit provided by Bender MedSystems) measurements, and antibody forming, cell count were performed according to methods known to the skilled artisan (see “Medical Immunology Experiment,” Publisher: People Hygiene, Published in 1999 in China, Editor: Xi Chuan Ping). Five days before the end of the treatment, 12 mice from each group (each replicate for 2 mice) were immunized with 0.5 ml of 2% sheep RBC (equivalent to −2×10⁸ cells per ml) for testing spleen B-cells. The relative weights of spleen and thymus to body weight (mg/100 g BW) were also measured. All statistical analyses were performed on ANOVA.

After conducting all tests and analyses, the relative weight of certain immune organs, namely the thymus and spleen, were measured, as illustrated in Table 4.

TABLE 4
Measurement of Organ Weight (mg/100 g of body weight)

| Organ | Control | Treatment 1 | Treatment 2 |
|---|---|---|---|
| Thymus | 534 ± 5.04 ^{C} | 549 ± 1.89 ^{B} | 567 ± 7.18 ^{A} |
| Spleen | 290 ± 6.33 | 298 ± 5.03 | 296 ± 7.50 |

Note:
Different superscript means statistically significant difference
“^{A}” and “^{B}” or “^{B}” and “^{C}” means P < 0.05
“^{A}” and “^{C}” means P < 0.01

The WBC count, lymphocyte transformation rate (specifically in the spleen), antibody forming cell, and natural killer cell killing rate (based on the killing rate of K562 cells by natural killer cell) were measured and analyzed, as illustrated in Tables 4and5.

TABLE 5
Analyses of Treatments

| Item | Control | Treatment 1 | Treatment 2 |
|---|---|---|---|
| WBC Count (10⁹/L) | 6.78 ± 0.54 | 7.12 ± 0.65 | 7.53 ± 0.55 |
| Lymphocyte Trans- | 1.10 ± 0.05 ^{B} | 1.15 ± 0.02 ^{B} | 1.24 ± 0.05 ^{A} |
| formation (SI | | | |
| Value, Stimula- | | | |
| tion Index) | | | |
| Antibody Forming | 1.44 ± 0.23 ^{A} | 1.48 ± 0.19 ^{A} | 0.91 ± 0.51 ^{C} |
| Cell (OD, Optical | | | |
| Density) | | | |
| NK Killing Rate (%) | 90.29 ± 2.24 | 92.19 ± 2.20 | 93.60 ± 2.21 |

TABLE 6
Plasma IL-2 and IL-6 Measurements

| Item | Control | Treatment 1 | Treatment 2 |
|---|---|---|---|
| IL-2 (pg/ml) | 6.05 ± 0.44 | 5.88 ± 0.53 | 5.76 ± 0.50 |
| IL-6 (pg/ml) | 247.83 ± 49.40 | 226.20 ± 37.93 | 247.00 ± 51.76 |

Lymphocyte transformation (or SI Value) is provided using CPM (Count per minute) of PHA containing tube/CPM (Count per minute) of tube without PHA. PHA-Phytohemagglutinin can stimulate inactive T-cell to convert to parental cell, which will eventually convert to active T-cell. In this regards, the stimulating index reflects the amount of inactivate T-cell available for turning into active T-cell. Thus, with a higher index, more inactive T-cells are present.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for affecting a patient's lymphatic system comprising identifying a patient who requires treatment for the lymphatic system; and administering to the patient an effective amount of a cysteamine compound to improve the patient's immunological response.

2. The method of claim 1, wherein the affected lymphatic system is selected from the group consisting of: lymphatic organs, mucosa membranes, goblet cells, and lymphocytes.

3. The method of claim 2, wherein the lymphatic organs are selected from the group consisting of a thymus or a spleen, wherein the method retards the deterioration in lymphatic organ mass.

4. The method of claim 2, wherein the lymphatic organs are selected from the group consisting of a thymus or spleen, wherein the method maintains lymphatic organ mass.

5. The method of claim 2, wherein the affected lymphatic system is mucosa membranes, wherein the method increases growth in villi along the mucosa membranes.

6. The method of claim 2, wherein the affected lymphatic system is goblet cells, wherein the method increases goblet cell activity.

7. The method of claim 2, wherein the affected lymphatic system is the lymphocytes, wherein the method enhances lymphocyte activity.

8. The method of claim 1, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

9. The method of claim 8, wherein said cysteamine salt is cysteamine hydrochloride or cysteamine phosphate.

10. The method of claim 9, wherein said cysteamine compound is taken orally, parenterally, intravenously, intramuscularly, transdermally, via buccal route, subcutaneously, or via suppository.

11. The method of claim 1, further comprising the step of concurrently administering to the patient at least one additional therapeutic agent.

12. The method of claim 11, wherein the therapeutic agent is selected from the group consisting of corticosteroids; cytotoxic drugs; non-cytotoxic drugs; nonsteroidal anti-inflammatory drugs; COX-2 inhibitors; antimalarials; plasma exchange; high-dose ivIg therapy; intravenous gamma globulin; and monoclonal antibody (moAb) therapy.

13. The method of claim 12, wherein the therapeutic agent is selected from the group consisting of: prednisone; hydrocortisone; azathioprine; cyclophosphamide; mycophenolate mofetil; methotrexate; ciclosporin; tacrolimus; ibuprofen; naproxen; celecoxib; rofecoxib; and hydroxychloroquine.

14. The method of claim 1, further comprising the step of diagnosing a patient with an immunological disorder.

15. The method of claim 1, wherein the effective amount of the cysteamine compound administered to the patient is between about 0.1 to 3,000 mg/kg of body weight or an equivalent molar quantity.

16. The method of claim 15, wherein the effective amount of the cysteamine compound administered to the patient is between about 1 mg/kg of body weight to 30 mg/kg of body weight of the cysteamine compound or an equivalent molar quantity.

17. The method of claim 15, wherein the effective amount of the cysteamine compound administered to the patient is between about 4 mg/kg of body weight to 18 mg/kg of body weight of cysteamine hydrochloride, or an equivalent molar quantity thereof.

18. A method for treating stress in a patient comprising identifying in the patient any one or more of the following: (a) symptoms of stress; (b) complications associated with stress; and (c) indications that the person is at-risk for stress; and administering to the patient an effective amount of a cysteamine compound.

19. The method of claim 18, wherein said cysteamine compound is selected from the group consisting of cysteamine, cystearnine salts, prodrugs of cysteamine, analogs of cysteamine, derivatives of cysteamine, conjugates of cysteamine, and metabolites of cysteamine.

20. The method of claim 19, wherein said cysteamine salt is cysteamine hydrochloride or cysteamine phosphate.

21. The method of claim 20, wherein said cysteamine compound is taken orally, parenterally, intravenously, intramuscularly, transdermally, via buccal route, subcutaneously, or via suppository.

22. The method of claim 18, further comprising the step of concurrently administering a therapeutic agent with the cysteamine compound, wherein the therapeutic agent is a therapy used to treat stress.

23. The method of claim 22, wherein the therapy is selected from the group consisting of counseling; psychotherapy; exercise; meditation; and massage therapy.

24. The method of claim 18, wherein the effective amount of the cysteamine compound administered to the patient is between about 0.1 to 3,000 mg/kg of body weight or an equivalent molar quantity.

25. The method of claim 24, wherein the effective amount of the cysteamine compound administered to the patient is between about 1 mg/kg of body weight to 30 mg/kg of body weight of the cysteamine compound or an equivalent molar quantity.

26. The method of claim 24, wherein the effective amount of the cysteamine compound administered to the patient is between about 4 mg/kg of body weight to 18 mg/kg of body weight of cysteamine hydrochloride, or an equivalent molar quantity thereof.
